(19) United States
(12) Patent Application Publication
(10) Pub. No.: US 2005/0090777 A1
(43) Pub. Date: Apr. 28, 2005

(54) Surgical irrigation system

(76) Inventor: Travis S. Carter, Kennewick, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/691,901

(22) Filed: Oct. 23, 2003

(51) Int. Cl. A61M 1/00

(52) U.S. Cl. 604/118

(57) ABSTRACT

A single-use surgical irrigation system for use with a surgical tool and including a sleeve to be slidably received over the tool, a fluid reservoir for storing a irrigation and cooling liquid, a tube mounted on the sleeve for conducting fluid from the reservoir to a dispensing tip at an opposing end of the tube, the dispensing tip configurable to aim the stream of ejected fluid in one or a plurality of directions. A flow control mechanism controls the volume of fluid flowing from the nozzle portion.

### TECHNICAL FIELD

The present invention pertains to devices for irrigating surgical wounds and tools, and, more particularly, to a single-use device with adjustable nozzle and flow control for removable attachment to a surgical tool, to a surgical irrigation system that includes a compressible fluid reservoir, and to a surgical instrument that combines the surgical irrigation system with a surgical tool.

### BACKGROUND OF THE INVENTION

Most bone cutting in foot and hand surgery is performed with a sagittal saw 10 or an oscillating saw 12, as shown in FIGS. 1A-1B. These saws are designed to be lightweight for handheld use. Typically, they are coupled to a source of power via a conduit, cable, or wire appropriate to the power source. The tools 10, 12 depicted in FIGS. 1A-1B have an elongated body portion 14 that is gripped by the user's hand. The two saws 10, 12 differ in that the sagittal saw 10 15 has a blade 16 that moves orthogonal to the plane of the reciprocating blade 18 of the oscillating saw 12.

Because the saw blades move at a high speed in order to cut through the dense bone material, substantial friction and heat are generated at the surgical site, both in the bone tissue and in the saw blades 16,18. Higher-powered saws are provided to accommodate geometric bone cuts, which can generate higher amounts of heat.

Cutting through the bone tissue will destroy some cells; however, the heat caused by the cutting operation kills additional bone cells on the end of the bone tissue, which is known as thermal necrosis (death of bone cells). The destruction of bone cells on the ends of the bone can slow healing and knitting of the bone back together and in some cases prevent a union of the bone tissue.

In order to reduce thermal necrosis, most surgeons will attempt to reduce the heat during sawing by applying a cooling liquid, as shown in FIG. 2. Here, the sagittal saw 10 being held by the surgeon's hand 20 has the saw blade 16 cutting into bone tissue 22. To provide access to the bone tissue 22, the wound 24 is held open by retractors 26 held in position by a surgical assistant. A second surgical assistant uses a syringe 32 to drop saline solution 30 onto the wound 24 and the saw blade 16. This method has the disadvantages of obscuring the field of vision at the wound site 24 and inconsistently applying the saline solution 30, resulting in the generation of heat in the bone tissue 22.

Attempts have been made to overcome the disadvantages of prior methods by utilizing powered pumps. One system designed for burring tools utilizes a rigid metal nozzle clamped to the tip of the burring tool and hooked to a motorized pump controlled by a foot pedal. Fluid is applied directly on the burring tool. System pressure is adjusted by a knob on a remote control panel for the pump. The disadvantage here is that the controls are located outside the sterile operative field. Consequently, a non-sterile assistant must be used to control the fluid pump. In addition, the nozzle clip is adaptable only to one size of tool, and the nozzle cannot be adjusted to direct the flow of fluid as desired. The foregoing system also requires expensive power equipment to push the saline solution through the system. While an I.V. bag can be used to allow gravity to push the saline through the system, both methods require a portion of the irrigation system to be contained outside of the sterile operative field. The I.V. bag and the supporting pole are not sterile. The motor to push the saline through the system is also not sterile. In addition, these systems are bulky, awkward, expensive, and can interfere with the handling of the surgical saw.

Another proposed system is described in U.S. Pat. No. 2,012,886 for dental hand pieces wherein a squeezable bulb containing fluid is manipulated With hand pressure to project water from a spray tube through a tapered end and onto a grinding wheel. Here, the entire hand must be used to squeeze the bulb. In U.S. Pat. No. 6,030,356, an irrigation clip is attached to a malleable metal tube for manually directing the flow of irrigation fluid near the cutting tip of a rotatable tool implement. Here, repeated bending of the metal tube can result in kinking, which will restrict the flow of fluid, and eventually metal fatigue will cause failure of the tube, resulting in leakage and eventually complete fracture.

### SUMMARY OF THE INVENTION

The disclosed embodiments of the invention are directed to a surgical irrigation device having an adjustable nozzle and flow control for removable attachment to a surgical tool, to a surgical irrigation system that includes a compressible fluid reservoir, and to a surgical instrument that combines a surgical tool with the surgical irrigation system.

In accordance with one embodiment of the invention, a surgical irrigation device is provided for use with a surgical tool that includes means for retaining and dispensing fluid; means for conducting the fluid from the dispensing means to the surgical wound; means for removably attaching the conducting means to the surgical tools; and means for controlling flow of fluid through the conducting means.

In accordance with another embodiment of the present invention, a surgical irrigation device for use with a surgical tool is provided. The device includes a sleeve attached to the tool; a conduit having a distal tip and a proximal end, the conduit mounted on the sleeve; and a flow control mounted on the sleeve and the conduit. Ideally, the sleeve is formed of expandable, compliant material to be received on the tool.

In accordance with another aspect of the foregoing embodiment, the conduit comprises a tube formed of flexible material, and the distal tip is formed of a positionable nozzle portion.

In accordance with a further aspect of the foregoing embodiment, the nozzle portion is formed of two or more nozzles, each nozzle individually adjustable to a desired position.

In accordance with another aspect of the foregoing embodiment, the flow control comprises a clamping member that is configured to enable selective clamping of the conduit or tube to control volume flow to the nozzle portion. Ideally, the clamping member is configured to enable selective clamping for stepped or proportional volume flow control.

In accordance with another embodiment of the invention, a surgical irrigation system for use with a surgical tool is provided. The system includes a sleeve sized and shaped to be slidably received on the surgical tool; a conduit mounted on the sleeve; a flow control mounted on the sleeve and attached to the conduit; and a reservoir coupled to the conduit. Ideally, the conduit has a distal dispensing tip formed at a distal end that can be positioned at a desired orientation.

In accordance with another aspect of the foregoing embodiment, the distal tip is formed of flexible non-metal material having a piece of malleable metal attached thereto to enable selective positioning of the dispensing tip at a desired orientation that will retain its position.

In accordance with another aspect of the foregoing embodiment, the reservoir comprises a compressible bladder, such as an elastomeric bulb formed of a flexible membrane, or a collapsible housing or shell, or a syringe. Means are provided for collapsing or compressing the reservoir, such as an elastic, resilient band.

In accordance with another embodiment of the invention, a surgical instrument is provided that includes a surgical tool having a cylindrical body and a surgical irrigation system mounted on the tool. The surgical irrigation system is preferably a single-use system that includes a sleeve formed of compliant material and sized to be slidably received over the cylindrical body of the tool. A fluid reservoir comprising a compressible container is attached to a tube mounted on the sleeve, the tube having a first end configured for dispensing the fluid from the fluid reservoir and a second end connectable to the fluid reservoir. The first end of the tube includes a nozzle portion formed of flexible material to enable selective positioning of the nozzle for directing fluid therefrom at a desired location, such as on the wound itself, on the tool or a blade associated with the tool, or both. A fluid control mechanism is mounted on the sleeve and associated with the tube for controlling the volume of fluid passing through the tube to the nozzle portion. The flow control mechanism is positioned ideally on the tool to enable operation thereof by the user's hand, preferably one or two fingers of the user's hand.

As will be readily appreciated from the foregoing, the present invention provides a surgical irrigation device and system that provides irrigation of the wound and cooling of the surgical instrument that is contained entirely within an operative field. The system is easily adaptable to a variety of instruments, is controlled at the instrument itself, arid provides adjustment by the user while holding the tool both directionally and in volume. The use of bags, poles, or power equipment is not required inasmuch as the fluid reservoir is compressed using elastomeric materials. Hence, the system lends itself to inexpensive fabrication, ease of use, and is readily disposable.

### BRIEF DESCRIPTION OF THE DRAWINGS

The foregoing and other features and advantages of the present invention will be more readily appreciated as the same become better understood from the following description when taken in conjunction with the accompanying drawings, wherein:

FIGS. 1A-1B are illustrations of known saws for use in surgical procedures;

FIG. 2 is an illustration of a surgical site showing the use of a sagittal saw in conjunction with a known irrigation method;

FIG. 3 is an isometric illustration of a surgical instrument system in disassembled configuration formed in accordance with the present invention;

FIG. 4 is an isometric illustration of another embodiment of a surgical instrument system utilizing the surgical irrigation device of the present invention; and

FIG. 5 is a side view of a clamp member formed in accordance with the present invention.

### DETAILED DESCRIPTION OF THE INVENTION

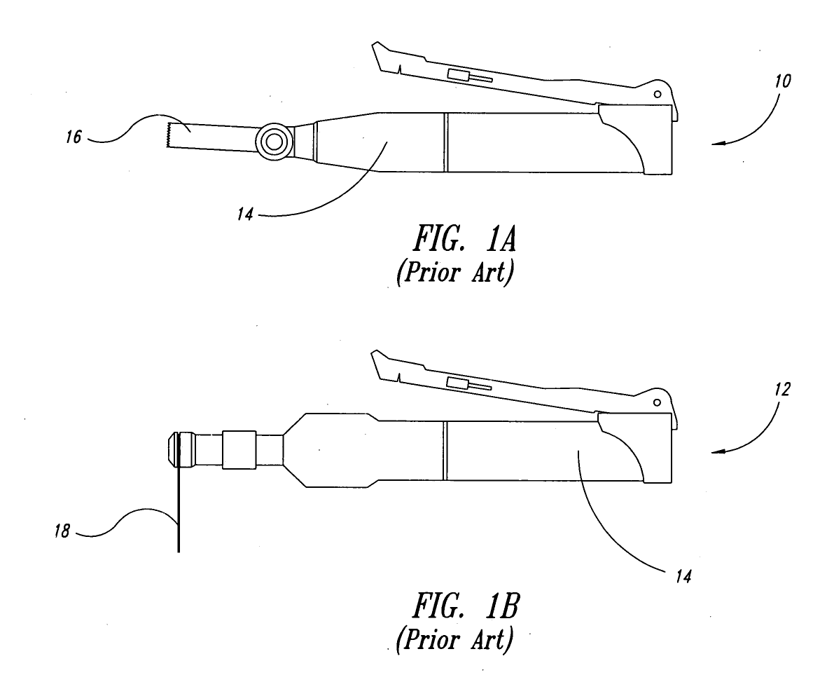

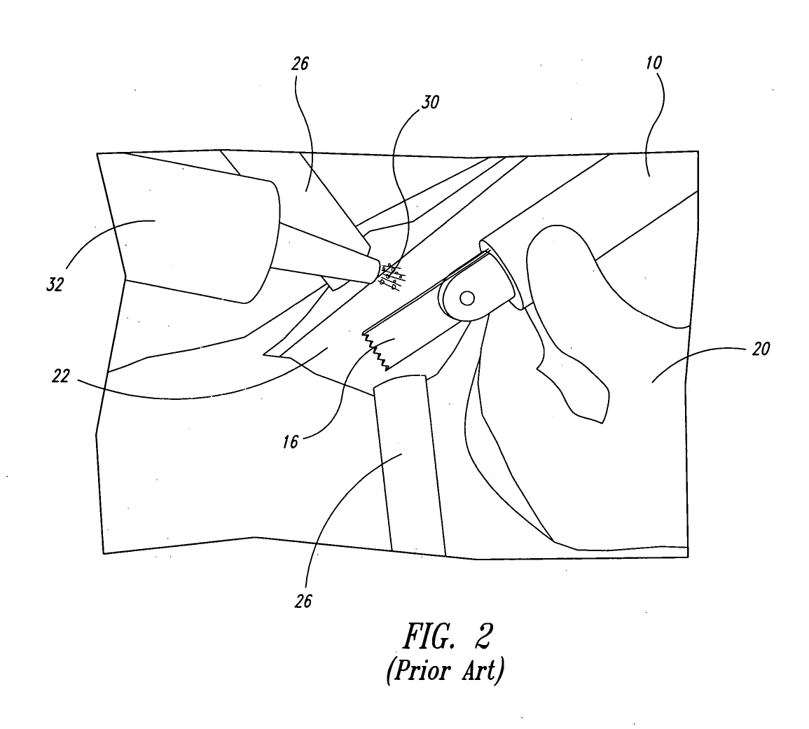

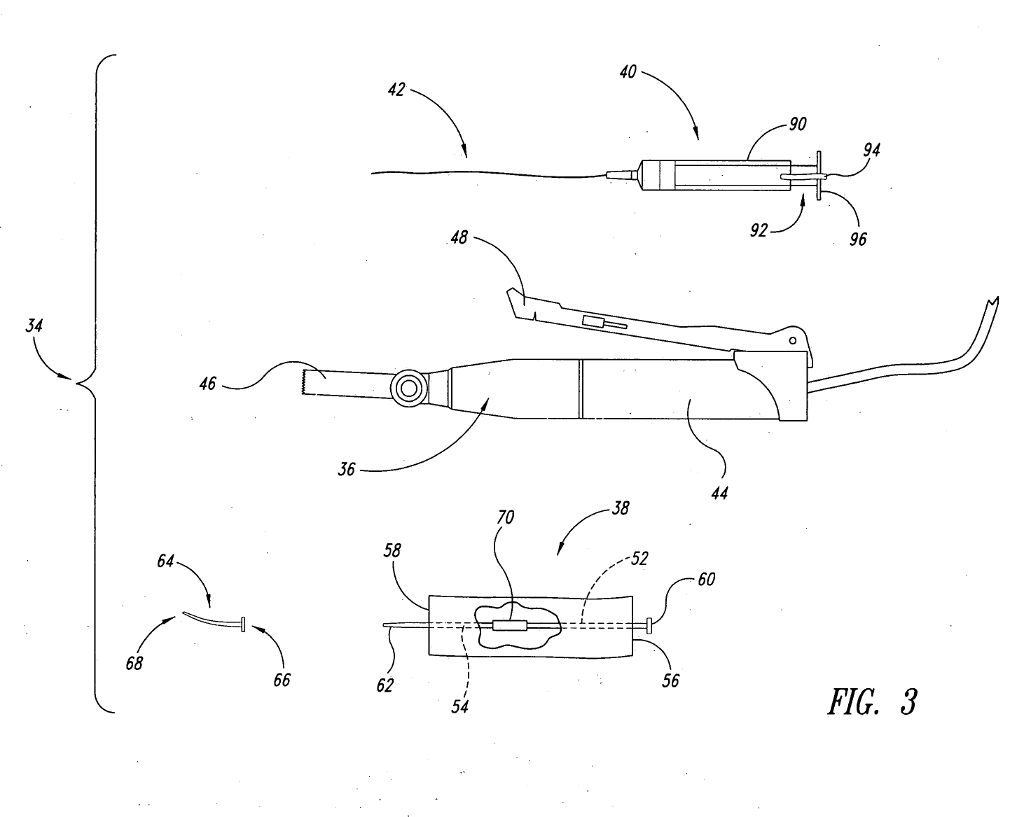

Referring initially to FIG. 3, shown therein are the components of a surgical instrument system 34 formed in accordance with one embodiment of the present invention. The system 34 includes a surgical tool, in this case a surgical saw 36, along with a surgical irrigation device 38. The system 34 further includes a syringe 40 attached to a thin, narrow tube 42. The surgical saw 36 is of the sagittal type, similar to the sagittal saw 10 of FIG. 1A, and includes the body portion 44, which is substantially elongate and cylindrical, and the saw blade 46. This tool is readily commercially available and will not be described in more detail herein. It is to be understood that other tools may be used in the system 34, including an oscillating saw, burrs, and other tools well known to those of skill in the art. This particular tool 36 includes an optional hand switch 48 pivotally attached to the body 44 to enable a user to activate the tool 36 with a single finger. A power cord 50 is detachably coupled to the tool 36 to provide power, such as pneumatic power, although electric or hydraulic power may be used as well.

The surgical irrigation device 38 includes a sleeve 52 that is preferably formed of elastic material that contracts and stretches so as to be compliant with the shape of the body 44 of the tool 36. Ideally the sleeve 52 is sized so as to be stretched over the body 44 to engage the body and provide a non-slip gripping surface for the user. Suitable material for the sleeve includes latex or similar material, including textured or non-slip material to enhance grip, as is known to those in the art.

In this particular embodiment, the sleeve 52 has a conduit or tubular member 54 integrally formed therewith for conveying fluid from a proximal end 56 to a distal end 58 of the sleeve 52. Ideally the tubular member 54 is integrally formed with the sleeve, although it may be attached to the exterior or interior of the sleeve. While the tubular member 54 may be separately formed from the sleeve 52, and then either slid under the sleeve 52 after it is installed on the tool 36 or first placed on the body 44 of the tool 36 and then the sleeve 52 slid thereover, such is not preferred because of the difficulty in positioning the sleeve 52 with respect to the tube and the tool 36. A connector 60 is formed at the end of the tubular member 54 adjacent the proximal end 56 of the sleeve 52 to facilitate connection and disconnection of the tubular member 54 from a fluid source. Typically, most tools can be uncoupled from the power cord, and this connector 60 provides a means for uncoupling the tubular member 54 from the fluid source when the power cord 50 is uncoupled.

The tubular member 54 terminates at a distal end 62 that is configured to receive a nozzle portion 64, shown separately in this depiction of the system 34. The nozzle portion 64 has a connection end 66 for coupling to the distal end 62 of the tube 54 and a dispensing tip 68. In this embodiment, the nozzle portion 64 is preferably formed of malleable material that enables it to be bent without kinking or failing from fatigue and to retain its position. The nozzle portion 64 is sized to extend over the cutting member of the tool 36, in this case the saw blade 46. While some surgeons may prefer to aim the stream of fluid at the saw blade, others will prefer to aim it at the bone itself. Thus, the ability to reposition the nozzle portion 64 to the desired location while accommodating differing needs and applications.

The nozzle portion 64 may be formed to have different configurations. For example, the tip can be perforated to provide a shower-like effect from a plurality of small streams of fluid. The nozzle portion 64 may also be dividable, such as having two conduits removably attached together to enable separation and individual positioning as desired, e.g., on both sides of the saw blade 46, or simultaneously on the saw blade 46 and on the wound 24. In another embodiment, the nozzle portion 64 can be formed from the tubular member 54 wherein the tubular member 54 is formed of flexible material having a malleable wire attached thereto that positions and aims the tubular member 54. It can also be bent so that it does not interfere with the saw blade or the bone when the blade is fully inserted into the bone.

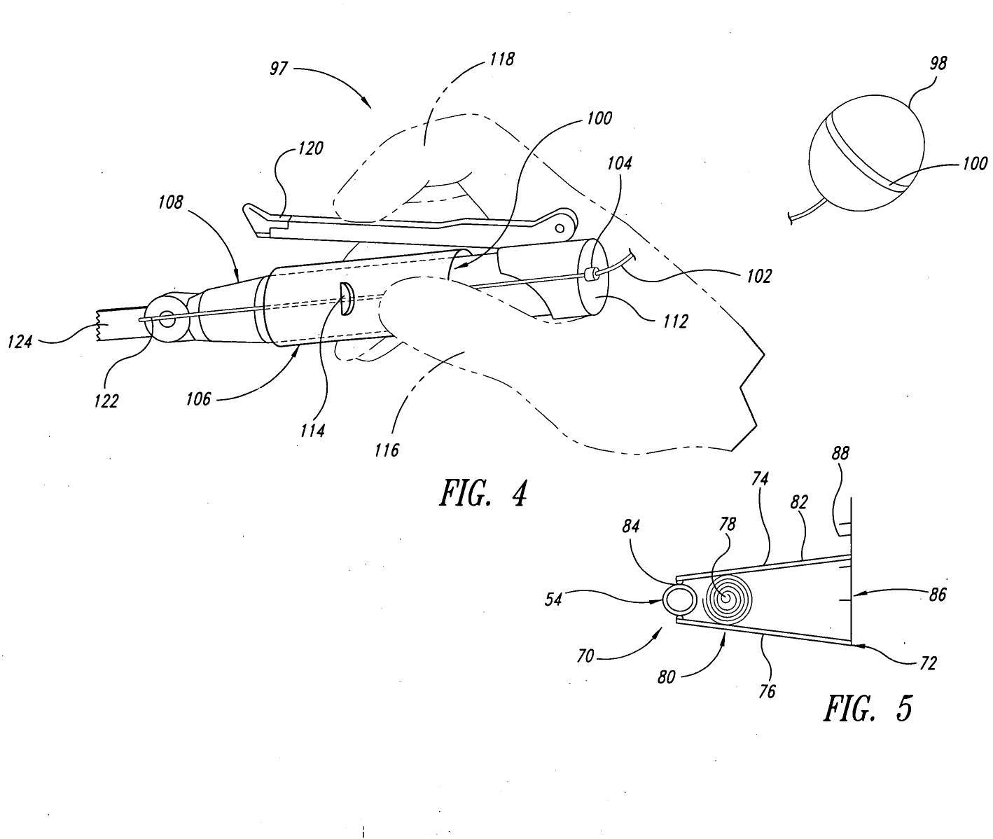

Still referring to FIG. 3, the surgical irrigation device 38 has includes a flow control mechanism 70 mounted on the sleeve and associated with the tubular member 54 to control the volume of fluid flowing through the tubular member 54. The flow control mechanism 70 is preferably integrally formed with the sleeve 52 and positioned so that it can be operated by the thumb of the user when the tool is in use, as described in more detail in connection with FIG. 4 below. The flow control mechanism 70 can have a number of configurations. In one embodiment, shown in FIG. 5, the flow control mechanism 70 consists of a clamp 72 having first and second clamping members 74, 76 coupled by a spring member 80 to pivot about a pivot point 78 and biased together by a spring member 80 to a closed position. When the clamp 72 is closed, the clamping members 74, 76 pinch the tubular member 54 closed to prevent the flow of fluid. Pressure applied to free the end 82 causes the engaged end 84 to separate from the second clamping member 76, allowing fluid to flow through the tubular member 54. A stop member 86 positioned near the free end 82 of the first clamping member 74 has detents 88 formed thereon that enable selective positioning of the degree of opening of the clamp 72. Thus, the free end 82 of the first clamping member 74 can snap into various positions and is held in place by the detents 88. In this embodiment, the stop member 86 is fixedly attached to the second clamping member 76.

Turing again to the surgical instrument system 34 of FIG. 3, the syringe 40 is of conventional configuration and is readily commercially available. Hence, it will not be described in more detail. Briefly, the syringe 40 includes a body 90 having a plunger 92 slidably received therein. The plunger 92 is urged to slide into the syringe body 90 by an elastic band 94 fixedly attached to the syringe body 90 and passing over the top 96 of the plunger 92.

Shown in FIG. 4 is another embodiment of a surgical instrument system 97 in an assembled state. This embodiment differs from the system 34 shown in FIG. 3 by the use of a compressible bladder 98 sized and shaped to contain the irrigation and cooling fluid, such as a saline solution. An elastic band 100 circumscribes the bladder to urge the bladder to collapse, thus applying pressure to the fluid inside the bladder, urging it to travel through the tube 102 to the connection member 104 associated with the surgical irrigation device 106 mounted to the surgical saw 108. The device 106 includes the sleeve 110 slidably received over the body 112 of the saw 108. A flow control mechanism 114 is formed as part of the sleeve 110 and positioned to be operable by the user's thumb (shown in phantom) 116 while the user's finger 118 (shown in phantom) operates the hand switch 120.

As shown here, the nozzle portion 122 is positioned adjacent the saw blade 124 to direct fluid from the compressible bladder 98 onto the saw blade 124.

Ideally, the surgical irrigation device 106 has a low profile and is formed of lightweight materials to facilitate handling and manipulation of the saw 108. The flow control mechanism 114 is preferably configured as a clamp, as described above for example with respect to FIG. 5, that opens and closes to control the flow of saline. The clamp is located where it can be easily pressed while sawing, and it can be positioned on the right side, left side, or bottom of the saw 108, depending on how the sleeve is slipped over the body 112 of the saw 108. This makes it possible to accommodate both right- and left-handed surgeons.

Thus, the disclosed embodiments of the invention provide an inexpensive, disposable method of irrigating a tool and an operating wound that remains within the operative field, is controllable by the user, does not need additional personnel to aid in its use, has a low profile, and does not require foot pedals or other accessories.

While a preferred embodiment of the invention has been illustrated and described, it is to be understood that various changes may be made therein without departing from the scope of the invention. For example, the flow control mechanism can consist of a roller, thumbscrew, or other known device for restricting the flow of fluid within a tube. And while the sleeve has been described as the preferred method for attaching the tube to the tool, it is to be understood that adhesive or other materials could be used, although this is not preferred because of the residue the adhesive would leave on the tool and its tendency to attract dirt, dust, and other foreign-objects. In addition, elastic bands can be used to attached the tube and flow control mechanism to the tool, which would enable selective positioning of the bands to accommodate the shape and construction of the tool, and which would lower the cost of the system and enhance its disposability.

In accordance with another embodiment of the invention, the tubular member 54 shown in FIG. 3 can be externally mounted to the sleeve 52 in a manner that permits adjustment in the positioning of the tubular member with respect to the sleeve, ideally along a longitudinal axis of the sleeve, to enable selective positioning of the nozzle portion 64 with respect to the saw blade 46 and the tool 36.

Hence, the invention is to be limited only by the scope of the claims that follow and the equivalents thereof.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1. A surgical irrigation device for use with a surgical tool, comprising:
a sleeve configured for attachment to the tool;
a conduit having a distal tip and a proximal end, the conduit mounted on the sleeve; and
a flow control mounted on the sleeve and associated with the conduit for controlling fluid flow through the conduit.

2. The device of claim 1 wherein the sleeve is formed of expandable, compliant material.

3. The device of claim 1 wherein the conduit comprises a tube formed of flexible material, and the distal tip comprises a positionable nozzle portion.

4. The device of claim 3 wherein the nozzle portion comprises at least two nozzles, each of the at least two nozzles configured to be individually adjustable to a desired position and to retain the desired position.

5. The device of claim 3 wherein the flow control comprises a clamping member.

6. The device of claim 5 wherein the clamping member is configured to enable selective clamping of the tube to control volume flow to the nozzle portion.

7. The device of claim 1, further comprising a connector at the proximal end of the conduit.

8. The device of claim 1, wherein the sleeve is formed of textured material to enhance gripping of the device.

9. A surgical irrigation device for use with a surgical tool, the device comprising:
means for holding and releasing fluid;
means for conducting fluid from the fluid holding and releasing means;
means for releasably attaching the conducting means to the tool; and
means for controlling flow of fluid through the fluid conducting means.

10. The device of claim 9 wherein the controlling means comprises means for clamping the conduit means to restrict the volume of fluid flowing through the conduit means.

11. The device of claim 9 wherein the fluid holding means comprises a compressible reservoir and means for compressing the compressible reservoir.

12. The device of claim 9 wherein the conducting means comprises a means for dispensing fluid out of the conducting means, the dispensing means configured to be selectively positionable to a desired position and to retain the desired position.

13. A surgical irrigation system for use with a surgical tool, comprising:
a sleeve sized and shaped to be slidably received onto the surgical tool;
a conduit mounted on the sleeve;
a flow control mounted on the sleeve and associated with the conduit; and
a reservoir coupled to the conduit.

14. The system of claim 13 wherein the conduit comprises a dispensing tip formed at a distal end thereof that can be positioned at a desired orientation.

15. The system of claim 14 wherein the dispensing tip is formed of malleable material.

16. The system of claim 14 wherein the dispensing tip comprises two nozzles that can be individually positioned at a desired orientation.

17. The system of claim 13 wherein the flow control comprises a clamp that squeezes the conduit to control volume flow through the conduit.

18. The system of claim 13, further comprising a connector coupled at a proximal end of the conduit to releasably connect the conduit to the reservoir.

19. The system of claim 13 wherein the reservoir comprises a compressible member.

20. The system of claim 19 wherein the compressible member comprises an elastomeric bulb urged into a compressed condition by an elastomeric member.

21. The system of claim 19 wherein the reservoir comprises a syringe.

22. The system of claim 19 wherein the reservoir comprises a collapsible bladder.

23. The system of claim 19 wherein the reservoir comprises means for compressing the reservoir.

24. The system of claim 23 wherein the collapsing means comprises a resilient band.

25. The system of claim 19 wherein the conduit is adjustably mounted on the sleeve to enable selective positioning of the conduit with respect to the sleeve.

26. The system of claim 19, wherein the sleeve is formed of textured material to enhance gripping.

27. A surgical instrument, comprising:
a surgical tool; and
a surgical irrigation device attached to the surgical tool, the surgical irrigation device comprising:
a sleeve attachable to the tool,
a conduit mounted on the sleeve, and
a flow control mounted on the sleeve and associated with the conduit.

28. The instrument of claim 27 wherein the sleeve is formed of expandable, compliant material.

29. The instrument of claim 27 wherein the conduit comprises a tube formed of flexible material, and the tube further comprises a distal tip having a positionable nozzle portion formed thereon.

30. The instrument of claim 29 wherein the nozzle portion comprises two nozzles, each nozzle individually adjustable to a desired position.

31. The instrument of claim 29 wherein the flow control comprises a clamping member.

32. The instrument of claim 31 wherein the clamping member is configured to enable selective clamping of the tube to control volume flow to the nozzle portion.

33. The instrument of claim 27, further comprising a connector at a proximal end of the conduit.

34. A surgical instrument system, comprising:
a surgical tool; and
a surgical irrigation system coupled to the tool, the surgical irrigation system comprising:
a sleeve sized and shaped to be slidably received onto the surgical tool,
a conduit mounted on the sleeve,
a flow control mounted on the sleeve and associated with the conduit, and
a reservoir coupled to the conduit.

35. The system of claim 34 wherein the conduit comprises a dispensing tip formed at a distal end thereof that can be positioned at a desired orientation.

36. The system of claim 35 wherein the dispensing tip is formed of malleable material.

37. The system of claim 35 wherein the dispensing tip comprises two nozzles that can be individually positioned at a desired orientation.

38. The system of claim 34 wherein the flow control comprises a clamp that squeezes the conduit to control volume flow through the conduit.

39. The system of claim 34, further comprising a connector coupled at a proximal end of the conduit to releasably connect the conduit to the reservoir.

40. The system of claim 34 wherein the reservoir comprises a compressible member.

41. The system of claim 40 wherein the compressible member comprises an elastomeric bulb urged into a compressed condition by an elastomeric member.

42. The system of claim 40 wherein the reservoir comprises a syringe.

43. The system of claim 40 wherein the reservoir comprises a collapsible bladder.

44. The system of claim 40 wherein the reservoir comprises means for compressing the reservoir.

45. The system of claim 44 wherein the collapsing means comprises a resilient band.

46. The system of claim 40 wherein the conduit is adjustably mounted on the sleeve to enable selective positioning of the conduit with respect to the sleeve.

47. A single-use surgical irrigation system for use with a surgical saw having a cylindrical body to provide fluid for irrigation and cooling, the system comprising:
a sleeve formed of compliant material and sized to be slidably received over the cylindrical body on the tool and to retain its position on the cylindrical body;
a fluid reservoir comprising a compressible container;
a tube mounted on the sleeve, the tube having a first end configured for dispensing fluid and a second end connectable to the fluid reservoir, the first end comprising a nozzle portion that is bendable to enable select positioning of the nozzle portion for directing fluid therefrom at a desired location; and
a fluid control mechanism mounted on the sleeve and associated with the tube for controlling the volume of fluid passing through the tube from the reservoir to the nozzle portion, the fluid control mechanism positioned on the tool to enable operation of the fluid control mechanism by one or more fingers of a user while holding the tool.

48. The system of claim 47 wherein the tube is adjustably mounted on the sleeve to enable selective positioning of the tube with respect to the sleeve to enable repositioning of the nozzle portion with respect to the tool.

49. The system of claim 47 wherein the nozzle portion comprises two nozzles, each nozzle selectively positionable.

50. The system of claim 47 wherein the fluid control mechanism comprises a clamp associated with a flexible portion of the tube and configured to squeeze the tube and control the volume of fluid flowing therethrough.

51. The system of claim 47 wherein the reservoir comprises a syringe having an elastic band attached thereto to apply pressure on a plunger in the syringe.

52. The system of claim 47 wherein the reservoir comprises an elastomeric bulb and an elastic band positioned on the elastomeric bulb to apply compressive pressure to the elastomeric bulb.

53. The system of claim 47 wherein the nozzle portion comprises a malleable wire attached to a flexible portion of the tube.

54. The system of claim 47 wherein the sleeve is formed of material to enhance gripping of the tool.
